(19) United States
(12) Patent Application Publication
(10) Pub. No.: US 2008/0021439 A1
(43) Pub. Date: Jan. 24, 2008

(54) Intradermal cellular delivery using narrow gauge micro-cannula

(75) Inventors: John M. Brittingham, Wake Forest, NC (US); John A. Mikszta, Durham, NC (US); Dominique J. Williams, Raleigh, NC (US); Cheryl H. Dean, Raleigh, NC (US)

Correspondence Address: BECTON, DICKINSON AND COMPANY;(ALSTON & BIRD LLP), 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 11/705,192

(22) Filed: Feb. 12, 2007

Related U.S. Application Data
(63) Continuation of application No. 10/758,274, filed on Jan. 16, 2004, now abandoned.
(60) Provisional application No. 60/504,488, filed on Sep. 19, 2003.
(60) Provisional application No. 60/440,348, filed on Jan. 16, 2003.

(51) Int. Cl. A61M 5/31

(52) U.S. Cl. 604/522

(57) ABSTRACT

A method for delivering cells into a subject by administering cells into the intradermal space of the skin of the subject by a microneedle. The cells are associated with cellular based therapeutics and vaccines and delivered by perpendicular insertion of the microneedle. The microneedle is a hollow needle having an exposed height of between about 0 and 1 mm, a total length of between about 0.3 mm to 2.5 mm, and a size of equal to or less than 30 gauge. An array of microneedles can also be used.

This application is a continuation application of U.S. application Ser. No. 10/758,274, filed Jan. 16, 2004, which claims the benefit of U.S. Provisional Applications Nos. 60/440,348, filed Jan. 16, 2003, and 60/504,488, filed Sep. 19, 2003, all of which are incorporated by reference in their entirety.

### FIELD OF THE INVENTION

The present invention relates to a method for delivering cellular based therapeutics and vaccines into subjects, particularly, for delivering dendritic cell or related cell type based therapeutics, islet cells, and vaccines into the intradermal space of the skin of the subjects by a microneedle.

### BACKGROUND OF THE INVENTION

Cellular based therapeutics and vaccines refer to treatments that use cells and tissues as therapeutic agents to treat injury or disease. Examples of cellular based therapeutics include, but are not limited to, hematopoietic cell therapeutics, mesenchymal stem cell based therapeutics, immunotherapies, dendritic cell and related cell type based therapeutics, and islet cell therapies. Islet cell therapies are based on the function of these cells to produce insulin to treat diabetes. Dendritic cell and related cell type therapy are based on the function of dendritic cells as antigen-presenting cells.

Dendritic cells originate in bone marrow and migrate into the thymus, and have both class I and class II of major histocompatibility complex (MHC) molecules on the surface. Dendritic cells are important vectors and antigen-presenting cells in the induction of an effective immune response against infection and neoplastic disease. Antigens alone, even those pre-processed to bind to antigen-presenting MHC class I and II molecules, are insufficient to regulate effective T-cell mediated immunity. Activated dendritic cells are essential to this task. Foreign antigens are displayed on the surface of these specialized antigen-presenting cells and enter the lymph node. One type (interdigitating dendritic cells) presents the antigen to T cells in the paracortical area of the lymph node, another type (follicular dendritic cells) is thought to be involved in activating memory B cells in the activated center (the germinal center) of lymphoid follicles.

Langerhans cells are related dendritic cells of the skin that play a key role in cutaneous immune response. Langerhans cells are dendritic precursor cells in the skin and are considered as sentinels standing guard against external stimuli. Langerhans cells reside in the basal and suprabasal layers of the epidermis and form a network of dendrites, through which they interact with adjacent keratinocytes and nerves. Langerhans cells are mobile, and they migrate to the T cell dependent area of lymph nodes. Like the macrophages, they are also bone-marrow derived, constitutively express MHC-II, and have potent antigen presenting properties. Unlike the macrophages, however, Langerhans cells have the ability to sensitize naive T cells.

Dendritic cell based therapies and vaccines and related cell type based therapeutics and vaccines usually require culture and activation of the dendritic cells outside of the patients (ex vivo), though the dendritic cells may be initially obtained from the same patients autologously. Activated dendritic cells having desired antigen on the surface are then re-introduced into the patient's body to regulate the immune response of the body.

The skin is a target for delivery of cellular based therapeutics and vaccines. The skin is the ultimate vessel for the human body: it receives and transports, accepts and expels according to the body's needs. It is the container, defender, regulator, breather, feeler, and adapter. The skin is the largest organ of the body and is as indispensable as the body's other major organs. Skin is made up of two primary layers that differ in function, thickness, and strength. From outside to inside, they are the epidermis and its sublayers, and the dermis, after which is the subcutaneous tissue, or the hypodermis. Epidermis and dermis are further differentiated by their respective amounts of hair follicle, pigmentation, cell formation, gland make-up, and blood supply. The total thickness of the skin varies from person to person and varies on a person according to the location of the skin on the body but is typically around 2-3 mm.

The epidermis layer is the outmost layer of the skin and has five layers. These layers are the stratum corneum, stratum lucidum, stratum granulosum, stratum spinosum, and stratum germinativum. The epidermis layer can be 5-30 cell layers thick depending on the age and the sex of the person and the location of the skin on the body. The total thickness of epidermis is typically between about 50 to about 150 microns.

The dermis layer underneath the epidermis layer has roles of regulating temperature and supplying the epidermis with nutrient-saturated blood. The dermis layer is made up of fibroblasts, which produce collagen connective tissue and lend elasticity and support to the skin. The dermis is the seat of hair follicles, nerve endings, and pressure receptors and defends the body against infectious invaders that can pass through the thin epidermis. The dermis layer is also subdivided into two divisions: the papillary dermis and the reticular layer. The papillary dermis is the main agent in dermis function for the supply of nutrients to selected layers of the epidermis and regulation of temperature. Both functions are accomplished with a thin but extensive vascular system that operates like vascular systems throughout the body. The reticular layer is much denser than the papillary dermis; it strengthens the skin, providing structure and elasticity. As a foundation, it supports other components of the skin, such as hair follicles, sweat glands, and sebaceous glands.

Methods for administering cellular based therapeutics and vaccines, particularly, dendritic cell and related cell type based therapeutics and vaccines, into the patients have been poorly studied. One method of administration is by intravenous infusion. See U.S. Pat. No. 6,077,519 (Storkus et al.), U.S. Pat. No. 5,846,827 (Celis et al.), U.S. Pat. No. 4,844,893 (Honsik et al.), and U.S. Pat. No. 4,690,915 (Rosenbery). The method of intravenous infusion of the cellular based therapeutics and vaccines, especially those therapeutics and vaccines targeted at the immune system including the lymph node, had disadvantages. First, these cellular based therapeutics and vaccines do not have direct access to the immune system, as they are circulating in the blood before they reach the lymph system. Second, very high cell numbers are required in order to provide an effective therapy. Third, in some cases, the intravenous delivery may induce a state of immune tolerance rather than activation.

The use of dendritic cells (DC) for immunotherapy of cancer and infectious diseases is a growing field. For cancer therapy, autologous DC are typically purified from a patient by leukophoresis, then are loaded with tumor material such as defined tumor antigens, tumor peptides, lysed tumor cells or tumor derived RNA (reviewed by M. Onaitis et al., Surg Oncol Clin N Am 11:645-660, 2002). These “loaded-DC” are then re-introduced to the patient in order to stimulate a specific immune response against the tumors.

The manner by which the DC are re-introduced to the patient has been a topic of considerable interest recently. A number of delivery routes have been investigated in pre-clinical animal studies and human clinical trials, including subcutaneous (SC), intradermal (ID), intravenous (IV), intraperitoneal (IP), intralymphatic (IL) and combinations of the above routes. Despite these studies, it is still unclear as to which route(s) will be most effective at preventing or treating cancer. Fong et al. showed that patients immunized with antigen-pulsed DC via ID, IL or IV routes all generated specific immune responses, although the quality and nature of the immune response differed among the routes (L. Fong et al., J. Immunol. 166:4254-4259, 2001). In particular, interferon (IFN)-γ producing T cell responses were observed following ID and IL but not IV delivery, whereas patients injected IV showed a greater propensity to generate tumor-specific antibodies. Efficacy (e.g., tumor reduction and/or prevention) was not assessed in this study. In another study by the same group, IV delivery was shown to be clinically efficacious in a subset of patients with advanced colorectal or non-small-cell lung cancer, although alternate routes were not investigated (L. Fong et al., Proc Natl Acad Sci, USA, 98:8809-8814, 2001).

A number of investigators have performed ID delivery of DC for cancer therapy (e.g., J S Yu et al., Cancer Res 61:842-847, 2001; Oosterwick-Wakka et al., J. Immunotherapy 25:500-508, 2002; T Azuma et al., Int J Urology 9:340-346, 2002; A E Chang et al., Clin Cancer Res 8:1021-1032, 2002; M Smithers et al., Cancer Immunol Immunother 52:41-52, 2002). In these studies, the method of ID delivery was either not specified or was performed according to the Mantoux technique using a standard needle and syringe. ID injections by the Mantoux technique are performed by inserting a needle (typically around 27 Ga) at a shallow angle to the skin surface (Flynn et al., Chest 106:1463-1465, 1994). This method is very difficult to perform even in the hands of highly trained practitioners and is often associated with pain. In addition, it is very difficult to control delivery depth according to this method, thus resulting in “spillover” of at least a portion of the administered dose into the SC tissue. In the prior art studies listed above, complete or partial clinical responses were observed only in a limited number of subjects in a subset of these studies (A E Chang et al., Clin Cancer Res 8:1021-1032, 2002; M Smithers et al., Cancer Immunol Immunother 52:41-52, 2002).

It is unclear as to whether improved ID delivery (i.e., to reduce or eliminate “spillover” of dose to SC tissue and provide more reproducible depth control across subjects) would improve clinical efficacy. Through trafficking studies in a limited number of human subjects, Morse et al. (M Morse et al., Cancer Res 59:56-58, 1999) suggest that DC injected IV localize to the lungs and then the liver, spleen and bone marrow but not the lymph nodes or tumors. Likewise, SC-injected DC did not traffic to the lymph nodes. In contrast, a small percentage of ID-injected DC in this study migrated rapidly to the lymph nodes. Although it is unclear whether better lymph node targeting will improve DC vaccine efficacy in humans, studies in mice have suggested that delivery routes that target the lymphatics are more effective at preventing or treating cancer than those that do not (A A O Eggert et al., Cancer Res 59:3340-3345, 1999; N Okada et al, British J of Cancer, 84:1564-1570, 2001).

Although delivery route has been the subject of considerable study and debate, there have been no reported studies investigating the potential effects of other delivery parameters on cell therapy, including, for example: cell concentration, flow rate, delivery volume or needle geometry (e.g., gauge size and needle length). These parameters have varied widely in prior studies, thus making it impossible to ascertain the potential role of such parameters in cell delivery.

Thus, there is need for developing an effective and more efficient treatment and administration of the cellular based therapeutics and vaccines.

### SUMMARY OF THE INVENTION

The present invention provides a method for delivering cells into a subject. The subject can be a human patient or an animal. The method comprises a step of administering cells into the intradermal space of the skin of the subject by a microneedle. The cells are associated with cellular based therapeutics and vaccines. The cellular based therapeutics and vaccines include hematopoietic cell therapeutics, mesenchymal stem cell based therapeutics, immunotherapies, dendritic cell and related cell type based therapeutics and vaccines, and islet cell based therapeutics. The cellular based therapeutics and vaccines are delivered by perpendicular insertion of the microneedle into the intradermal space of the skin.

In the method of the present invention, the microneedle is a hollow needle having an exposed height of between about 0 and 1 mm and a total length of between about 0.3 mm to about 2.5 mm. Preferably, the microneedle is a hollow needle having a length of less than about 2.5 mm. Most preferably, the microneedle is a hollow needle having a length of less than about 1.7 mm. The cellular based therapeutics and vaccines are delivered into the skin to a depth of at least about 0.3 mm and no more than about 2.5 mm by the microneedle.

The microneedle used in the method of the present invention is preferably less than 27 gauge and more preferably between 50 gauge and 30 gauge. Most preferably, the microneedle is between 34 gauge and 30 gauge. In addition to a single microneedle, an array of microneedles can also be used in this invention.

The present invention also provides a method for curing or preventing diseases by administering cellular based therapeutics and vaccines into the intradermal space of the skin of a subject by a microneedle. The cellular based therapeutics and vaccines are selected from the group consisting of hematopoietic cell therapeutics, mesenchymal stem cell based therapeutics, immunotherapies, dendritic cell and related cell type based therapeutics and vaccines, and islet cell based therapeutics. The cellular based therapeutics and vaccines are delivered by perpendicular insertion of the microneedle into the intradermal space of the skin so that cellular based therapeutics and vaccines are delivered into the skin to a depth of at least about 0.3 mm and no more than about 2.5 mm.

The dendritic cells and other cells to be used in the method of the invention can be obtained and cultured by any suitable means familiar to those of skill in the art. For example, dendritic cells may be obtained by peripheral blood leukophoresis and density gradient centrifugation. The dendritic cells may be obtained from subjects that were treated with Flt3L to mobilize the dendritic cells prior to collection. Dendritic cells may be matured and activated in vitro using cytokines (for example, GM-CSF, IL-4, IFN-γ, TNF-α) before administration to the subject or may be administered as immature and non-activated cells.

The studies presented herein examined the effects of cell concentration, needle gauge, needle length and flow rate on the viability of a DC cell line and the expression of cell surface markers important for DC function.

### BRIEF DESCRIPTION OF DRAWINGS

FIG. 1 a shows the distribution of P815 cells following the intradermal delivery in pig skin taken by fluorescent microscope; the magnification is 10 times.

FIG. 1b shows the distribution of P815 cells following the intradermal delivery in pig skin taken by fluorescent microscope; the magnification is 20 times.

FIG. 2 shows the distribution of the fluorescent beads following the intradermal delivery in pig skin taken by fluorescent microscope. The bead diameter is 2.0 μm; the magnification is 20 times.

FIG. 3 shows the distribution and migration of the fluorescent beads following the intradermal delivery in pig skin taken by fluorescent microscope. The bead diameter is 2.0 μm; the magnification is 40 times.

FIG. 4 shows the distribution and migration of the fluorescent beads following the intradermal delivery in pig skin in greater details taken by fluorescent microscope. The bead diameter is 2.0 μm; the magnification is 60 times.

FIG. 5a shows the distribution of the fluorescent beads following the intradermal delivery in pig skin taken by fluorescent microscope. The bead diameter is 2.0 μm; the magnification is 20 times.

FIG. 5b shows the distribution of the fluorescent beads following the intradermal delivery in pig skin taken by fluorescent microscope. The image represents the tissue immediately below, and partially overlapping with, that presented in FIG. 5a. The bead diameter is 2.0 μm; the magnification is 20 times.

FIG. 6 shows the distribution and migration of the fluorescent beads following the intradermal delivery in pig skin taken by fluorescent microscope. The bead diameter is 0.027 μm; the magnification is 10 times.

FIG. 7 shows the distribution and migration of the fluorescent beads following the intradermal delivery in pig skin in greater details taken by fluorescent microscope. The bead diameter is 0.027 μm; the magnification magnification is 20 times.

FIG. 8 shows the uptake of fluorescent beads in the draining lymph nodes following the intradermal delivery in mouse skin at various times post-delivery. The bead diameters are 0.05 μm and 0.1 μm. Fluorescent beads were detected in the draining lymph nodes by flow cytometry.

FIG. 9 shows the uptake of fluorescent beads in the draining lymph nodes following the intradermal delivery in mouse skin at various times post-delivery. The bead diameters are 1 μm and 10 μm. Fluorescent beads were detected in the draining lymph nodes by flow cytometry.

FIG. 10 shows pressure profiles associated with delivery of JAWS DC cell line at differing concentrations delivered through various needles at 100 μl/min flow rate.

FIG. 11 show pressure profiles associated with delivery of JAWS DC cell line at differing concentrations delivered through various needles at 400 μl/min flow rate.

### DETAILED DESCRIPTION OF THE INVENTION

The present invention provides a method for curing or preventing diseases by delivering cells into the intradermal layer of the skin of a subject by a microneedle. The subject includes mammals generally and more specifically, humans. The cells are associated with cellular based therapeutics and vaccines, and can be either whole cells or transformed cells, or cellular components (e.g., membrane fragments, vesicles, exosomes, dexosomes).

The intradermal layer of the skin is an ideal target for the delivery of cellular based therapeutics and vaccines. The intradermal layer is abundant with both lymphatic drainage channels and dense capillary bed, which allow access to the blood circulation. Cellular based therapeutics and vaccines that target at lymphatic system and blood would benefit from proper delivery into the intradermal layer. For example, dendritic cell based therapeutics and vaccines need to have access to the lymphoid tissue where the antigen-specific immune responses can be initiated. Direct access to the lymphatic drainage system in the intradermal layer is a more effective way of administering dendritic cell based therapeutics and vaccines than through the conventional intramuscular or subcutaneous tissues.

Further, in the intradermal layer, nerve ends are located in a deeper layer inside the intradermal layer. Thus, the first insertion and delivery of the therapeutics and vaccines into the upper layer of the intradermal layer is preferred in the present invention, as the tips of the micro-cannula/microneedles would have no contact with the nerve ends and as a result, the patients sense no pain. To be effective, many of the dendritic cell based therapeutics and vaccines need to have access to the lymph node rather than the vascular system. In the present invention, the cellular based therapeutics and vaccines are delivered into at least about 0.3 mm to no more than about 2.5 mm under the surface of the skin. Preferably, the cellular based therapeutics and vaccines are delivered 0.5 mm to 2 mm under the surface of the skin. Intravenous (I.V.) delivery of the therapeutics and vaccines is less effective and efficient than delivery into the intradermal tissue. In some cases, I.V. delivery may even induce an immune tolerance in patients rather than activation.

Additionally, intradermal delivery of dendritic cell therapeutics and vaccines places the dendritic cells in a specialized microenvironment. Such a delivery method enables the dendritic cells to be placed in a microenvironment with the proper cytokines, chemokines, and other related factors to ensure the effective targeting of the lymph node and to remain activated in order to both stimulate naive resting T cells as well as re-activate memory T cells.

The method of the present invention is useful for cellular based therapeutics and vaccines which target the lymph system and vascular system, because intradermal delivery provides access to the lymph drainage system and the capillary system. Cellular based therapeutics and vaccines that can be used in the method of the present invention include, but are not limited to, hematopoietic cell therapeutics, mesenchymal stem cell based therapeutics, immunotherapies, dendritic cell and related cell type based therapeutics and vaccines, and islet cell based therapeutics.

Intradermal delivery of the therapeutics and vaccines is accomplished by perpendicular insertion of a microneedle, in a form of a micro-cannula, and preferably using depth-limiting features to restrict delivery to a given tissue depth. The delivery method is easier to perform than the conventional Mantoux technique and provides for more reproducible intradermal delivery with better control over depth of delivery.

The microneedle for the perpendicular insertion and intradermal delivery of the therapeutics and vaccines has a reduced diameter, shortened bevel length and shortened overall needle length as compared to conventional needles. The microneedle used in the delivery method of the present invention is a hollow needle having an exposed height of between about 0 and 1 mm and a full length of about between about 0.3 mm and about 2.5 mm. Preferably, the needle is less than about 2.0 mm. More preferably, the length is less than about 1.7 mm. The microneedle can be a single 30 gauge needle. Preferably, the microneedle is between 50 gauge and 30 gauge. More preferably, the microneedle is between 34 gauge and 30 gauge. An array of microneedles of the same size or varying sizes may be used. A properly designed array of microneedles would enable one to overcome the high pressure associated with the intradermal delivery in vivo. Each of the microneedles would have a configuration in accordance with the above description.

The cellular based therapeutics and vaccines can be stored in a reservoir connected to the microneedle before and during the delivery. An appropriate medium may also be included in the reservoir to keep the cellular based therapeutics and vaccines alive and activated for a desired period of time.

A conventional means for pumping the cellular based therapeutics and vaccines through the microneedle may be used. For example, a syringe commonly used in the healthcare industry with a suitable diameter may be hermetically connected to the microneedle or a mini pump may be used for this purpose. Alternatively, the cellular based therapeutics and vaccines may be hermetically sealed in a reservoir and can be pumped by anyone applying pressure on the reservoir. The microneedle and the means for pumping may be connected directly together or through some connecting means such as a catheter tube. The microneedle and the catheter tubing may be optionally coated with a polymer or other substances, e.g., serum proteins, to prevent or reduce the number of cells sticking to the surfaces, especially during slow-rate extended delivery.

The present invention for intradermal delivery of the cellular based therapeutics and vaccines has a number of advantages:

First, the present invention provides improved targeting of the lymphatic drainage system through the intradermal delivery. Because of this, therapy may be accomplished with fewer cells and dose reduction becomes possible. In addition, localized or systemic delivery of cells may be achieved depending on the desired therapy.

Second, the method of the present invention eliminates or reduces the need for leukophoresis, which is a purification step for the autologously obtained dendritic cells. Leukophoresis is typically performed on whole blood from patients in order to purify the autologous dendritic cells. Better targeting of the lymphatic drainage system combined with the delivery to the proper microenvironment for the dendritic cell maturation and activation may make such purification step unnecessary. The overall autologous therapies can be greatly simplified.

Third, the method of the present invention provides for systemic drug therapy via ID delivery of cells producing therapeutic protein(s); e.g., islet cells producing insulin may be delivered to the intradermal space to treat diabetics.

In one preferred embodiment of the invention therapeutic cells are delivered via a microinfusor or similar device that controls delivery rate and other biomechanical factors. For reproducible ID delivery, the device should comprise narrow gauge cannula (e.g., 30 Ga to 34 Ga) that are inserted perpendicular to the skin surface to a depth determined by the length of the needle and position of a depth limiting hub.

The following examples are illustrative, but not limiting the scope of the present invention. Reasonable variations, such as those occur to reasonable artisan, can be made herein without departing from the scope of the present invention.

### EXAMPLE 1

### Cell Viability Following Microneedle Delivery

Purpose:

Cells were delivered in vitro through a microneedle designed for intradermal delivery and tested for viability following such delivery.

Method:

1. Experimental materials.
a. Microneedle. A 34 gauge microneedle with a length of 1 mm length was used.
b. Cell line. P815 cell line was a mouse mastocytoma-derived cell line (Lundak, R L & Raidt, D J, Cell. Immunol. 9:60-66, 1973) that is often used as a model system for antigen-presentation studies. In particular, P815 cells could be transfected with genetic material encoding specific antigens such as those used in a vaccine for an infectious disease or cancer, or could be loaded with such antigens directly. Then, these P815 cells could be used to stimulate T cells in vitro.

P815 cells for this experiment had a size of 10-15 μm in diameter on non-adherent rounded cells, which was of a similar size to dendritic cells and related Langerhans cells. In this example, P815 cells were used as a model cellular therapeutic for in vitro study.

2. Experimental steps.
The following steps were followed in the experiment:
(1) Suspensions of P815 cells were made at various concentrations mimicking the concentrations used in many clinical settings. See Table 1.
(2) The cell suspensions were loaded into a 1 cc syringe which was connected to a 34 gauge 1 mm length stainless steel microneedle connected to an approximately 3-inch long catheter tube.
(3) Between 100 and 200 μl of cell suspension was delivered in approximately 5 to 10 seconds, at a rate typical for a rapid bolus style injection and flowed through the microneedles.

3. Recordation and assessment of results.

The microneedles were monitored by video microscopy. Cell viability was assessed by trypan blue staining before and after delivery. Percentage of viability was calculated based on the trypan blue staining results.

Results:

1. As indicated in the captured video microscopy, there was no cell clumping or occlusion of the microneedle during the delivery.

2. The viability of the cells before and after the delivery is indicated in Table 1.

TABLE 1
Viability of Cells

| Cell Concentration | Viability (%) | Viability (%) |
|---|---|---|
| (cells/ml) | Before Delivery | After Delivery |
| 20 × 10⁶ | 97 | 97 |
| 10 × 10⁶ | 97 | 97 |
| 5 × 10⁶ | 97 | 95 |
| 1 × 10⁶ | 94 | 91 |

As indicated in Table 1, there was no significant change in cell viability before and after the cells passed the microneedle.

Conclusion:

The model cellular based therapeutics and vaccines could be effectively passed through the microneedle in vitro without disrupting cell viability and causing cell clumping or occlusion of the microneedle according to the method of the present invention; similar results are expected in vivo. Similar results have also been obtained with other cell types including, e.g., an immortalized dendritic cell line (see Example 2, below), a hepatocellular carcinoma cell line (HepG2, as described in U.S. Pat. No. 4,393,133, Jul. 12, 1983) and a pancreatic tumor cell line (AR42J, as described by Jessop, N W & Hay, R J, In Vitro 16:212, 1980). Thus, one skilled in the art will appreciate that the present invention is applicable to various cell types of diverse characteristics.

### EXAMPLE 2

### Dendritic Cell Viability Following Microneedle Delivery

Purpose:

A dendritic cell line (JAWS-II, as described in U.S. Pat. Nos. 5,648,219, and 5,830,682) was delivered through two different types of microneedles designed for intradermal delivery (30 Ga and 34 Ga needles) and tested for viability and expression of functional cell surface markers following such delivery. Delivery through a standard 27 Ga needle was included for comparison.

Method:

1. Experimental materials.
a. Needles and Microneedles: Cells were delivered through: a) a standard 27 Ga needle, b) a 30 Ga, 1.5 mm long stainless steel microneedle or c) a 34 Ga, 1.0 mm long stainless steel microneedle.
b. Cell line. The mouse JAWS-II cell line, as described in U.S. Pat. Nos. 5,648,219 and 5,830,682 was used for these studies. Prior to delivery, cells were activated for 24 hr with IFN-γ, IL-4, TNF-α and GM-CSF, as described in U.S. Pat. Nos. 5,648,219 and 5,830,682.

2. Experimental steps.

The following steps were followed in the experiment:
(1) Suspensions of JAWS cells were made at various concentrations (20, 40 or 80 million cells/ml) mimicking the concentrations used in many clinical settings. See Table 2.
(2) The cell suspensions were loaded into a 1 cc syringe which was connected to a) a standard 27 Ga needle, b) a 30 Ga gauge 1.5 mm length stainless steel microneedle or c) a 34 Ga 1.0 mm length stainless steel microneedle connected to an approximately 3-inch long catheter tube.
(3) Approximately 200 μl of cell suspension was delivered at one of 3 different flow rates: a) hand-driven bolus delivery (ranging from approximately 3000-6000 μl/min for the 27 Ga and 30 Ga needles, and ranging from approximately 700-1000 μl/min seconds for the 34 Ga microneedles), b) 100 μl/min flow rate controlled by a Harvard syringe pump or c) 400 μl/min flow rate controlled by a Harvard syringe pump.

3. Recordation and assessment of results.

Cell viability was assessed by trypan blue staining and flow cytometry staining for 7-Amino-actinomycin D (7-AAD) before and after delivery.

Results:

The viability of the cells before and after the delivery is indicated in Table 2.

TABLE 2
Viability of Dendritic Cells

| Needle | Cell Concentration | Flow Rate | % Viability Trypan Blue | | | % Viability 7AAD | |
|---|---|---|---|---|---|---|---|
| Gauge | (cells/ml) | 200 ul delivered | Culture** | Control*** | Post-delivery | Control*** | Post-delivery |
| 27 | 20 × 10⁶ | Hand | 96 | 92 | 93 | 91 | 93 |
| | | 400 ul/min | 96 | 92 | 92 | 91 | 88 |
| | | 100 ul/min | 96 | 92 | 92 | 91 | 92 |
| | 40 × 10⁶ | Hand | 95 | 88 | 82 | nd | 94 |
| | | 400 ul/min | 95 | 88 | 94 | nd | 89 |
| | | 100 ul/min | 95 | 88 | 96 | nd | 91 |
| | 80 × 10⁶ | Hand | 95 | 82 | 91 | 82 | 83 |
| | | 400 ul/min | 95 | 82 | nd | 82 | nd |
| | | 100 ul/min | 95 | 82 | 84 | 82 | 81 |
| 30 | 20 × 10⁶ | Hand | 96 | 92 | 93 | 91 | 93 |
| | | 400 ul/min | 96 | 92 | 93 | 91 | 87 |
| | | 100 ul/min | 96 | 92 | 91 | 91 | 91 |
| | 40 × 10⁶ | Hand | 95 | 88 | 88 | nd | 92 |
| | | 400 ul/min | 95 | 88 | 91 | nd | 88 |
| | | 100 ul/min | 95 | 88 | 93 | nd | 91 |
| | 80 × 10⁶ | Hand | 95 | 82 | 86 | 82 | 79 |
| | | 400 ul/min | 95 | 82 | 86 | 82 | *78 |
| | | 100 ul/min | 95 | 82 | 88 | 82 | 81 |
| 34 | 20 × 10⁶ | Hand | 96 | 92 | 90 | 91 | 89 |
| | | 400 ul/min | 96 | 92 | 87 | 91 | 84 |
| | | 100 ul/min | 96 | 92 | 93 | 91 | 90 |
| | 40 × 10⁶ | Hand | 95 | 88 | 82 | nd | 86 |
| | | 400 ul/min | 95 | 88 | 74 | nd | 75 |
| | | 100 ul/min | 95 | 88 | 88 | nd | 88 |
| | 80 × 10⁶ | Hand | 95 | 82 | 74 | 82 | 70 |
| | | 400 ul/min | 95 | 82 | 85 | 82 | *77 |
| | | 100 ul/min | 95 | 82 | 62 | 82 | 66 |

*Single point value All other values are n = 2
**Culture: indicates cell viability directly out of culture; i.e., no passage through cannula and no storage on ice
***Control: indicates cell viability for cells that were kept on ice for the length of the deliver study but were not delivered through cannula
nd: not done

In these studies, JAWS cells at all 3 concentrations were delivered through the 27 Ga and 30 Ga needles at all 3 delivery rates with no observed occlusion of the cannula. In addition, there were no differences in cell viability, based on both trypan blue and 7-AAD staining, following delivery through the 27 Ga and 30 Ga cannula. Further, there were no differences in the expression of cell surface markers involved in DC function (CD54 and CD11c) following delivery through the 27 Ga and 30 Ga cannula. Thus, the 30 Ga microneedles as described in the present invention are as effective as standard 27a needles in delivering cells. Twenty-seven gauge needles are commonly used for intradermal injections according to the Mantoux-technique, but due to the extended length of the bevel and associated leakage of the dose out of the skin, are unable to be used according to the method of intradermal delivery whereby the needle is inserted perpendicularly to the skin surface. In addition, intradermal delivery according to the Mantoux technique using 27 Ga needles is often associated with spillover of the dosage into the SC tissue and patient pain. The 30 Ga needles of the present invention are designed for reproducible intradermal delivery controlled by the cannula length and position of a depth-limiting hub feature with no patient pain perception.

For the 34 Ga microneedles, some reductions in cell viability were observed under certain conditions. Generally, at all 3 cell concentrations at which reductions in cell viability were observed, there was a momentary occlusion of the microneedle; at 20 million cells/ml, ½ microneedles occluded at the 400 μl/min delivery rate. At 40 million cells/ml, 2/2 microneedles occluded at both the 100 μl/min and 400 μl/min flow rates. At 80 million cells/ml, ½ microneedles occluded in the group in which delivery was performed by hand, while the 400 μl/min rate tested had no observed occlusion. At the 100 μl/min rate, 2/2 microneedles occluded and only approximately 100 μl was delivered. Thus, as the concentration of cells increased from 20-80 million cells/ml, the % viability decreased for all delivery rates when administered through the 34 Ga microneedles.

Conclusion:

A dendritic cell derived cell line, JAWS II, is effectively delivered through 30 Ga microneedles with no resultant loss in cell viability or expression of cell surface markers with results similar to those obtained using conventional 27 Ga needles. The viability of these cells when administered through 34 Ga microneedles is dependent upon the cell concentration, whereby a reduction in cell viability is observed when the cell concentration is increased from 20-80 million cells/ml.

### EXAMPLE 3

### Cell Distribution Following Intradermal Delivery In Vivo

Purpose:

Characterize the distribution pattern of cells administered in vivo using microneedles.

Method:

1. Model system used.

Pigs were used for intradermal injection. Pig skin represents a well accepted model for human skin. P815 cells, as described in Example 1, were used as the model cell line.

2. Experimental steps.

P815 cells were delivered intradermally by 34 gauge microneedles 1 mm in length. Cells were suspended to a concentration of 40×10⁶ cells/ml. A total of 0.1 ml (4×10⁶ cells) was administered via bolus injection over a time period of approximately 1 minute.

3. Evaluation of results.

Immediately after allowing the bleb to resolve, full thickness skin biopsies were collected and processed for tissue sectioning and Haematoxylin & Eosin (H&E) staining. Pictures were taken from the light microscope observation.

4. Results

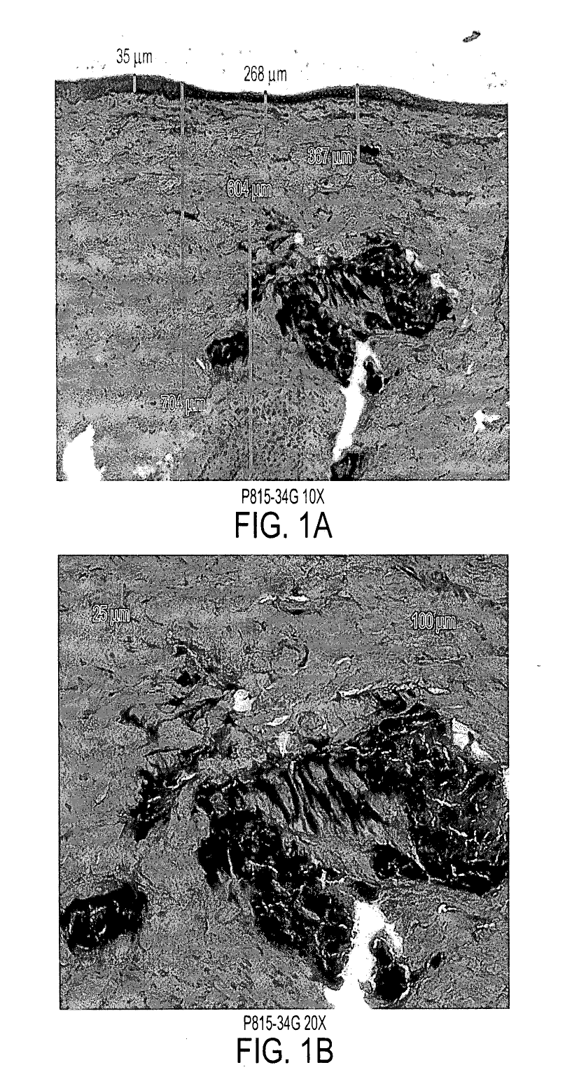

FIG. 1 displays the distribution pattern of P815 cells following intradermal delivery by the microneedle. Due to their high concentration and localized delivery, the P815 cells appear in the H&E stained image as darker and more tightly packed than the resident cells in the tissue. The distribution pattern illustrates delivery from a depth of about 0.3 mm to a depth of about 1.0 mm (FIG. 1a). In addition, cells were evident in what appear to be drainage channels spaced radially from the location of the bolus injection (see arrows in FIG. 1b).

5. Conclusion

The intradermal delivery method of the present invention was effective in delivering cells in vivo. The cells were delivered effectively through the microneedle and did not clump, rupture or occlude the microneedles. The ID delivery method of the present invention resulted in cells localized to the shallow ID tissue and there was evidence for rapid drainage and clearance of the cells from the delivery site. The shallow distribution of cells within the skin provided by microneedle delivery is not reproducibly achievable using 27 Ga needles and the Mantoux technique.

### EXAMPLE 4

### Direct Targeting of the Lymphatic Drainage Channels

Purpose:

The intradermal delivery of the cellular based therapeutics and vaccines of the present invention was tested in vivo for delivery efficiency and direct targeting of the lymphatic drainage channels in the skin.

Method:

1. Model system used.

Pigs were used for intradermal injection. Fluorescent beads of various sizes (0.027-15 μm range) were used for intradermal delivery and facilitated observation under microscope. The fluorescent beads of various sizes were used as surrogate markers for 20 cells of various sizes or cell derived components (e.g., membrane fragments, vesicles, exosomes, dexosomes). While therapeutic cells such as DC are typically within the range of about 10-50 μm in diameter, therapeutic cell-derived components such as membrane fragments, vesicles, exosomes and dexosomes are typically much smaller and within the range of about 0.05-2.0 μm in diameter.

2. Experimental steps.

The fluorescent beads were delivered intradermally by a 34 gauge microneedle 1 mm in length. Delivery of 100 μl volume was accomplished over a period of approximately 10 to 20 seconds using a microneedle affixed to 3 inch catheter line and 1 cc syringe.

3. Evaluation of results.

Thirty (30) minutes after the delivery, full thickness skin biopsies were performed and collected from the delivery sites and processed for H & E staining and fluorescent microscopy. Pictures were taken from the fluorescent microscope observation.

Results:

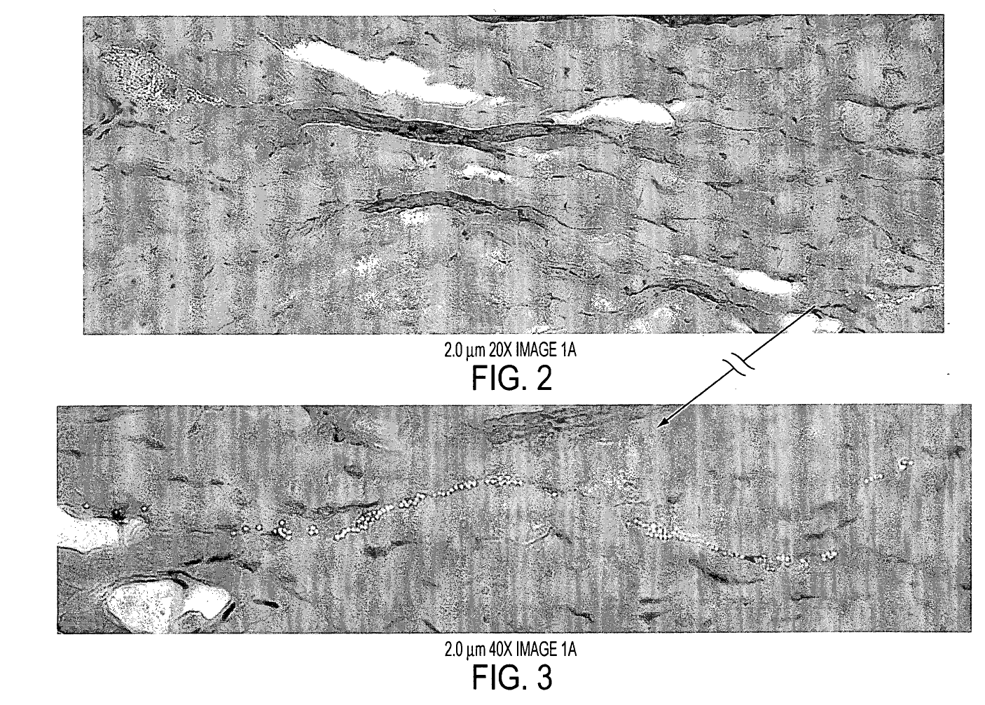

The results are shown in FIGS. 2-7.

In FIG. 2, putative needle insertion point and a track of the beads along the putative needle track are shown by the beads and the arrows. The bead diameter is 2.0 μm; the magnification is 20 times. Approximately 200,000 cells were administered.

FIG. 3 shows a concentration of beads in a typical bleb and linear track of beads radiating outward from the bleb. The bead diameter is 2.0 μm; the magnification is 40 times.

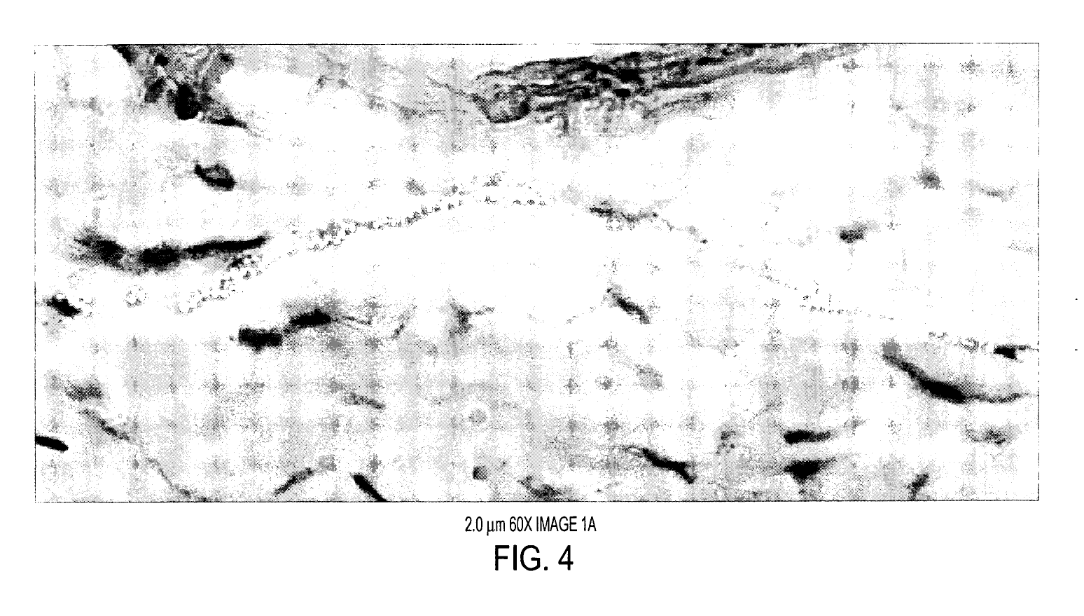

FIG. 4 shows in greater details than FIG. 3, an intradermal fluid bleb visible at the left, while a linear track of beads is present at the right, substantially distant from the intradermal bleb site. The bead diameter is 2.0 μm; the magnification is 60 times.

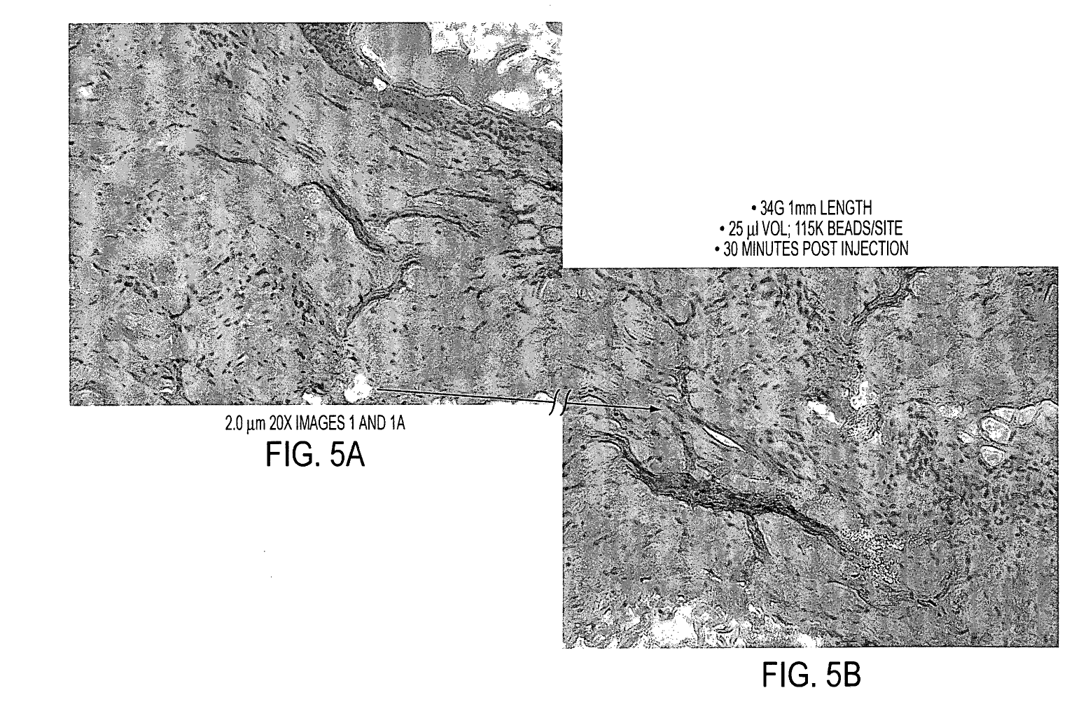

FIGS. 5a and 5b show the distribution of the beads in the intradermal layer after the delivery. FIG. 5b shows the proper target of the capillary system and the lymph drainage system within the intradermal layer by the beads. The bead diameter is 2.0 μm; the magnification is 20 times.

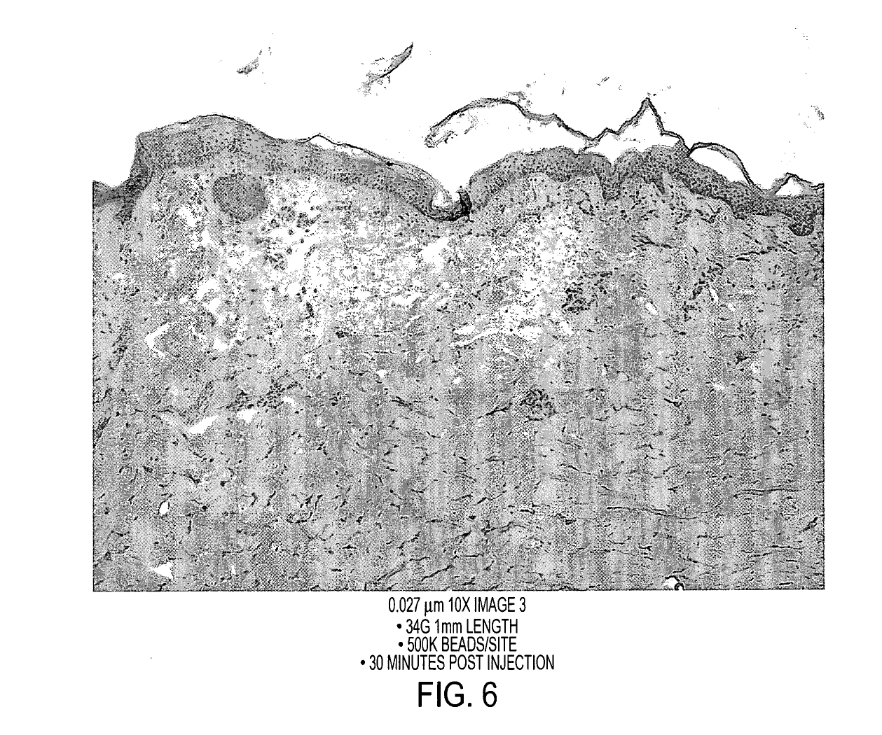

FIG. 6 shows the distribution of the beads in the upper layer of the dermis layer of the skin, the target area for lymph drainage system and capillary system. The epidermis layer is demarcated by the darker-stained, thin layer at the top. The bead diameter is 0.027 μm; the magnification is 10 times. Approximately 500,000 beads were administered.

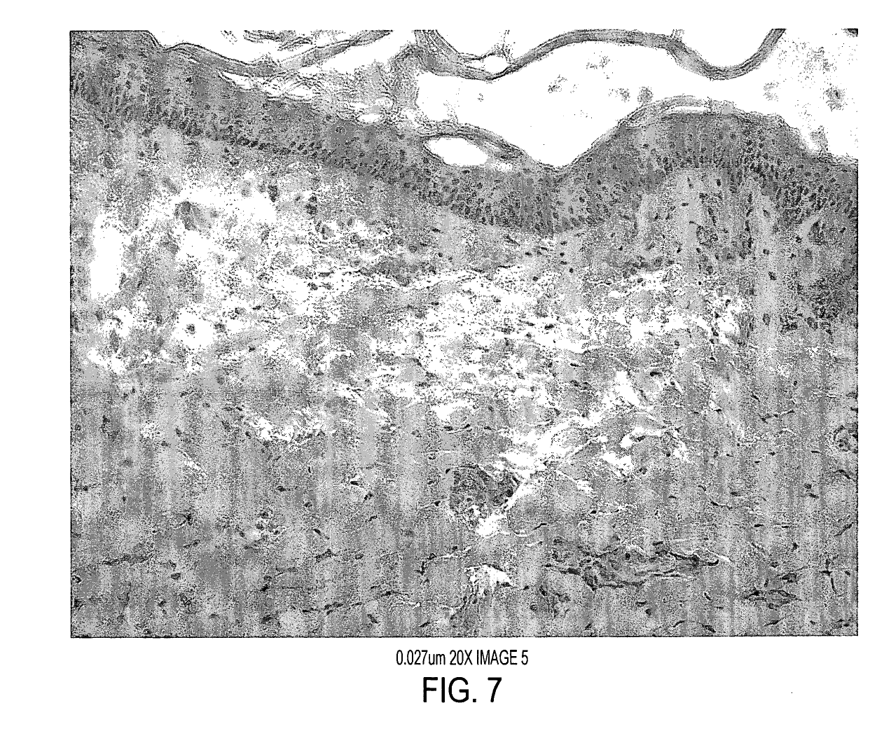

FIG. 7 shows the distribution of the beads in the upper layer of the dermis layer of the skin, the target area for lymph drainage system and capillary system in greater details. The bead diameter is 0.027 μm; the magnification magnification is 20 times.

Similar results with larger beads (10-15 μm) were also observed, suggesting that cell types within this size range would exhibit similar distribution patterns.

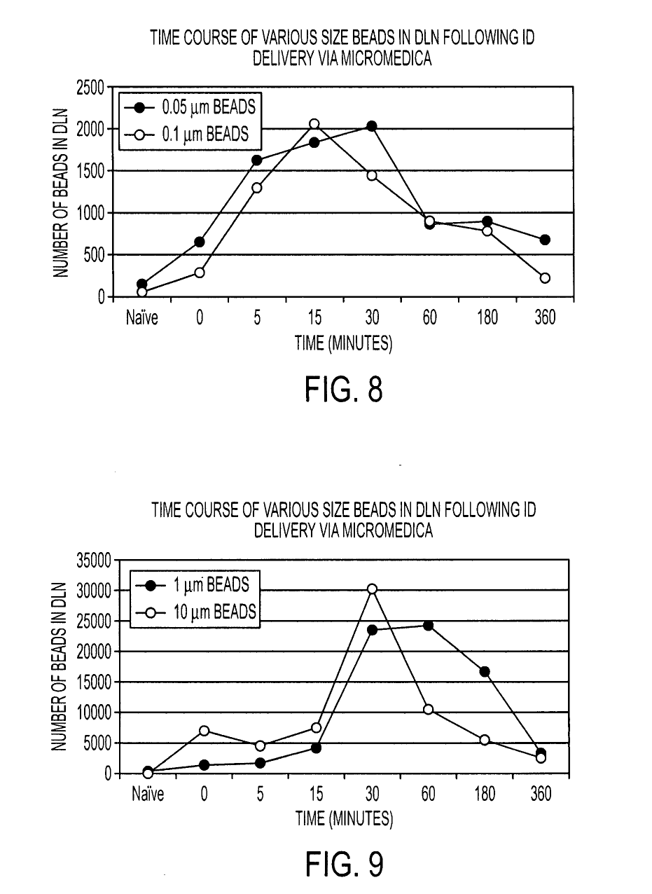

To demonstrate that the fluorescent beads target the draining lymph nodes (DLN) following intradermal delivery via microneedle, mice were injected with various size beads. FITC-labeled beads were injected ID using 34 Ga 1 mm length exposed needle into the lower dorsal region of C57BL/6 mice. 500,000 beads of 2 sizes (0.05 μm and 0.1 μm) were injected into both sides of the lower dorsal region 30 μl per side or 60 μl total per mouse (2 mice or 4 DLN per timepoint). At designated timepoints, the DLN were excised and a single cell suspension was prepared and sorted for FITC positive signal on the FACSVantage by sorting a 1.0 ml DLN suspension. Total counts of beads are on a per DLN basis. A standard curve composed of naïve DLN mixed with serially diluted bead numbers was generated for each bead size to determine the minimum signal detectable over background/autofluorescence. Both the 0.05 μm and 0.1 μm beads were observed in the DLN from the ID injection within minutes with a maximum reached in about 15 minutes (FIG. 8).

In a separate study, larger sized beads were examined. FITC-labeled beads were injected ID using 34 Ga 1 mm length exposed needle into the lower dorsal region of C57BL/6 mice. 1,000,000 beads of 2 sizes (1.0 μm and 10 μm) were injected into both sides of the lower dorsal region 30 μl per side or 60 μl total per mouse (2 mice or 4 DLN per timepoint). At designated timepoints, the DLN were excised and a single cell suspension was prepared and sorted for FITC positive signal on the FACSVantage by sorting a 1.0 ml DLN suspension. Total counts of beads are on a per DLN basis. A standard curve composed of naïve DLN mixed with serially diluted bead numbers was generated for each bead size to determine the minimum signal detectable over background/autofluorescence. Both the 1.0 and 10 μm beads were observed in the DLN from the ID injection within minutes with a maximum reached in about 30 minutes. There is a 15 minute shift from the smaller size beads indicating that as bead size increases, migration time to the DLN increases.

The previous examples demonstrate that the intradermal delivery method of the present invention is effective in delivering the beads, of a similar size to the cellular therapeutics and vaccines and cell-derived therapeutics and vaccines (e.g., membrane fragments, vesicles, exosomes, dexosomes), in vivo, in a fashion which facilitates distribution and did not cause clumping, rupturing of the beads, or the occlusion of the microneedle. Moreover, the intradermal delivery method of the present invention is effective for delivering the microbead to target area including the lymphatic drainage channels and DLN. In addition, the micro-bead experiments demonstrate that similar distribution and clearance patterns can be achieved using microparticles and nanoparticles, such as those commonly used in drug and vaccine formulations.

### EXAMPLE 5

### Pressure Profiles Associated with Cell Delivery Through Microneedles

This invention describes cell delivery methods for cellular therapy through various size cannula controlling such parameters as flow rate, cell concentration, and delivery volume. The limited success of current DC therapies may be due, at least in part, to the lack of consideration of these parameters in human clinical trials. The present invention describes methods to manipulate flow rate, cell concentration, delivery volume, and cannula size to improve cell viability and immunological function.

Cellular therapeutics, such as DC, typically range in size from about 10 μm to about 50 μm, although the actual size can vary substantially depending on the maturation/activation state of the cell and the extent of cell aggregation between cells. The standard cannula used for cell therapy are typically around 23-27 Ga, with inside diameter as presented in Table 3.

TABLE 3
Needle Dimensions

| Gauge | Cannula Length | O.D. (inches/μm) | I.D. (inches/μm) |
|---|---|---|---|
| 16 | 0.5 | 0.0655/1664 | 0.0485/1231 |
| 16 | 1 | 0.0655/1664 | 0.0485/1231 |
| 23 | 0.5 | 0.0255/648 | 0.0145/368 |
| 23 | 1 | 0.0255/648 | 0.0145/368 |
| 27 | 0.5 | 0.0165/419 | 0.0095/241 |
| 27 | 1 | 0.0165/419 | 0.0095/241 |
| 30 | 0.5 | 0.0125/318 | 0.0070/178 |
| 30 | 1 | 0.0125/318 | 0.0070/178 |
| 34 | 0.289 | 0.0070/178 | 0.0035/89 |
| 34 | 0.289 | 0.0070/178 | 0.0035/89 |

In the examples below, the various needles displayed in Table 3 were examined for pressure profiles associated with delivery of a DC line in vitro. The mouse DC line (JAWS II, CRL11904, ATCC, as above) was maintained in standard tissue culture conditions and then placed at three cell concentrations (80, 40, 20×10⁶ cells/ml) prior to delivery through the cannula at controlled flow rates and volumes via a Harvard syringe pump. Cell solutions were passed through an in-line pressure transducer.

Pressure Profiles for 100 μl/min Flow Rate

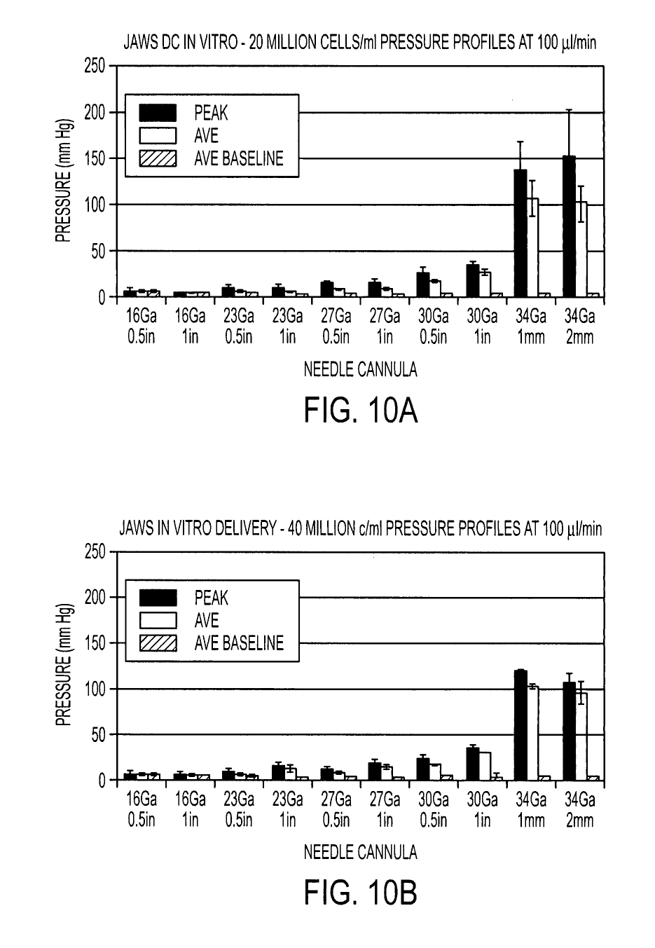

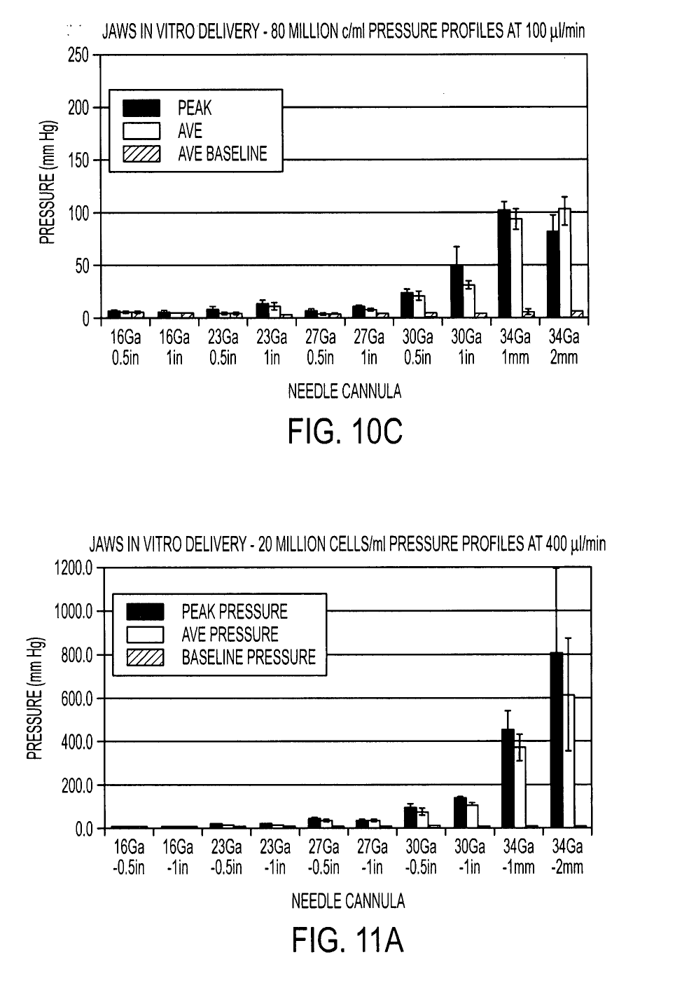

At the 100 μl/min flow rate, pressures increased with decreasing cannula diameter. Peak pressures were as low as about 5 mm Hg for the 16 Ga cannula and as high as about 100 mm Hg for the 34 Ga cannula (FIG. 10). Similar results were observed across all 3 cell concentrations.

Pressure Profiles for 400 μl/min Flow Rate

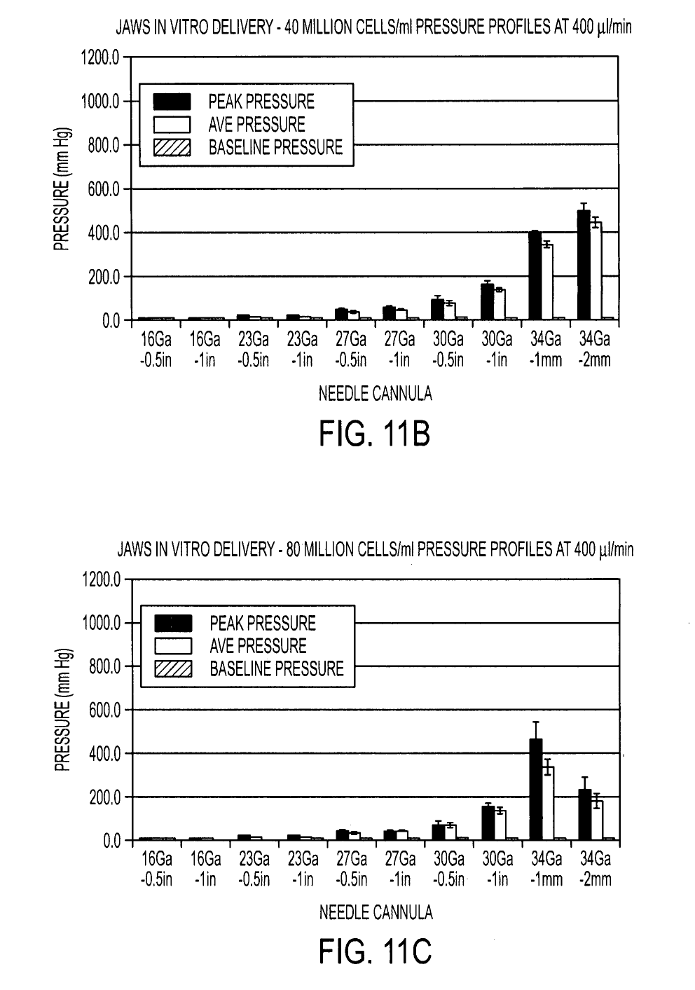

At the 400 μl/min flow rate, pressures also increased with decreasing cannula diameter. For the 16 Ga and 23 Ga cannula, there were no major differences in pressures observed at 400 μl/min as compared to 100 μl/min. For the 27 Ga, 30 Ga and 34 Ga cannula, however, there was a marked increase in pressure associated with delivery at the higher flow rate (FIG. 11); the higher flow rates were generally associated with an approximate 3-fold increase in peak pressure for these cannula, regardless of cell concentration. Peak pressures were as high as about 450 mm Hg for the 34 Ga cannula. The high pressures associated with delivery of cells through the 34 Ga microneedles may be associated, at least in part, with the reduction in cell viability observed under certain delivery conditions using these cannula (Table 2).

That which is claimed:

1. A method for delivering cells to a human or animal, said method comprising administering said cells intradermally through at least one small gauge cannula or needle wherein said cannula or needle is between 30 and 34 gauge, wherein the cells are administered in a concentration of 20-100 million cells/ml.

2. The method of claim 1 wherein said cannula or needle is 30 gauge.

3. The method of claim 1 wherein said cannula or needle is 34 gauge.

4. The method of claim 1 wherein said cells are dendritic cells.

5. The method of claim 4 wherein said cells are interdigitating dendritic cells.

6. The method of claim 4 wherein said cells are immature dendritic cells.

7. The method of claim 4 wherein said cells are mature dendritic cells.

8. The method of claim 1 wherein said cells are delivered into skin at a depth of 0.3 to 2.5 mm.

9. The method of claim 1 wherein said cells are delivered at a flow rate between 100 and 400 μl/min.

10. The method of claim 1 wherein said cells are delivered at a flow rate of less than 100 μl/min.

11. The method of claim 1 wherein said cells are delivered at a flow rate of greater than 400 μl/min.

12. The method of claim 3 wherein said cells are administered at a concentration of less than 80 million cells/ml.

13. The method of claim 3 wherein said cells are administered at a concentration of less than 40 million cells/ml.

14. The method of claim 3 wherein said cells are administered at a concentration of 20 million cells/ml.

15. The method of claim 1 wherein said animal is a mammal.
